(19) United States
(12) Patent Application Publication
(10) Pub. No.: US 2008/0208071 A1
(43) Pub. Date: Aug. 28, 2008

(54) Measurement of Gastric Acid Secretion

(75) Inventors: Anthony Axon, Harrogate (GB); Michael Clough, London (GB)

Correspondence Address: KIRTON AND MCCONKIE, 60 EAST SOUTH TEMPLE,, SUITE 1800, SALT LAKE CITY, UT, 84111, US

(73) Assignee: THE LEEDS TEACHING HOSPITALS NHS TRUST, Leeds (GB)

(21) Appl. No.: 11/995,461

(22) PCT Filed: Jul. 12, 2006
(86) PCT No.: PCT/GB2006/002580

§ 371 Date: Jan. 24, 2008

(30) Foreign Application Priority Data
Jul. 12, 2005 (GB) 0514194.0
Jan. 19, 2006 (GB) 0601079.7

(51) Int. Cl. A61B 5/00; C01B 31/00

(52) U.S. Cl. 600/532; 128/898; 423/419.1

(57) ABSTRACT

A method of measuring the amount of gastric secretion in a mammal involves administering a substance or formulation containing an excess quantity of a water-insoluble carbonate to the mammal to react with acid in the stomach, in which the insoluble carbonate is enriched with at least one isotope selected from ¹³C, ¹⁴C, ¹⁷O and ¹⁸O in a known amount. The content of the or each selected isotope in the exhaled carbon dioxide is allowed to stabilise before obtaining a sample of exhaled air containing carbon dioxide, and determining the content of the selected or each selected isotope in the exhaled carbon dioxide.

The present invention relates to a non-invasive method for the measurement of gastric acid secretion in mammals, including humans.

All mammals secrete gastric acid. Acid however is not necessary for the absorption of food, neither is it an essential for life. The reason acid secretion has been conserved is to create an acid barrier in the upper gastrointestinal tract to protect the rest of the digestive system from the pathogenic micro-organisms that may be present in food.

Acid secretion can lead to the development of duodenal ulcer, gastric ulcer and reflux oesophagitis. Conversely impaired or absent gastric acid secretion may predispose to the development of gastric cancer.

Drugs have been developed that can help to reduce the secretion of gastric acid. The proton pump inhibitor, omeprazole, has been widely prescribed for this purpose. Since then many other proton pump inhibitors have been marketed for the treatment of acid related disorders.

The mechanism of gastric acid secretion is complex involving psychological, neurological and hormonal control mechanisms. Secretion varies widely in individuals during the day dependent upon food intake and the ability to secrete acid also differs between individuals and populations.

The measurement of gastric acid secretion is difficult. A known method involves use of a naso-gastric tube through which acid is sucked from the stomach, while the same time giving a stimulus to excite secretion. This is an effective way to determine the maximal acid secretion in an individual but the invasive and unpleasant nature of the technique limits its applicability, both in research and clinical practice. The development of a non-invasive method is extremely desirable. This would enable epidemiological research, but even more importantly it would be possible to measure the effect of anti-secretory drugs in large numbers of subjects. This would enable the assessment of different anti-secretory drugs and their dosage. However, it is within the clinical arena that the value of a non-invasive investigation would be of particular value. Some patients fail to respond to treatment with anti-acid medications and it is unclear in these cases whether failure is a result of ineffective dosage, resistance to the drug being exhibited or whether failure to respond is because the disease process itself is not acid related.

A non-invasive method of measuring acid secretion would therefore be of considerable value in epidemiological research, in the development of more effective acid suppressive drugs, and in clinical management.

In one aspect, the invention provides a method of measuring the amount of gastric secretion in a mammal, the method comprising the steps:
a. administering a substance or formulation containing an excess quantity of a water-insoluble carbonate to the mammal to react with acid in the stomach, in which the insoluble carbonate is enriched with at least one isotope selected from ¹³C, ¹⁴C, ¹⁷O and ¹⁸O in a known amount,
b. allowing the content of the or each selected isotope in the exhaled carbon dioxide to stabilise,
c. obtaining a sample of exhaled air containing carbon dioxide, and
d. determining the content of the selected or each selected isotope in the exhaled carbon dioxide.

The method can be used to identify patients who (a) fail to respond adequately to acid suppressive treatment, (b) suffer from hypochlorhydria, (c) suffer from atrophic gastritis, and (d) have an increased risk of developing gastric cancer.

The insoluble carbonate may be administered on its own i.e. as a pure substance or mixture of substances without any excipients, or it may be part of a formulation containing conventional pharmaceutical excipients.

Preferably, the substance or formulation does not include any component other than the water-insoluble carbonate which reacts with acid in the stomach, for example in such a way as to increase pH. The formulation might include soluble components to facilitate ingestion, which might be in the form of covering layers or fillers and the like. Such components should not be capable of reacting with stomach acid, for example such that the pH in the stomach is increased significantly, for example as a result of a reaction between an acid and a base, or as a result of a hydrolysis reaction. An example of a component which might be included is gelatin which can be used to enclose a quantity of a carbonate powder in the form of a capsule.

In another aspect, the invention provides a pharmaceutically acceptable formulation containing a water-insoluble carbonate which can react with acid in a mammalian stomach, in which the insoluble carbonate is enriched with at least one isotope selected from ¹³C, ¹⁴C, ¹⁷O and ¹⁸O in a known amount, in which the formulation does not include any component other than the water-insoluble carbonate which reacts with acid in the stomach.

In a further aspect, the invention provides a use of an insoluble carbonate enriched with at least one isotope selected from ¹³C, ¹⁴C, ¹⁷O and ¹⁸O in a known amount in the preparation of a medicament for the treatment or prophylaxis of elevated levels of gastric acid.

In another aspect, the invention provides the use of an insoluble carbonate enriched with at least one isotope selected from ¹³C, ¹⁴C, ¹⁷O and ¹⁸O in a known amount in the preparation of a medicament for the detection of or management of pernicious anaemia.

Preferably, the method of the invention includes a step, prior to the step of administering the carbonate, of obtaining a sample of exhaled air containing carbon dioxide and determining the content of the selected or each selected isotope in the exhaled carbon dioxide.

The content of the selected isotope in exhaled carbon dioxide can be determined using techniques such as mass spectroscopic analysis, infra red spectroscopic analysis, laser assisted ratio analysis, gas chromatography with mass selective detector analysis, scintillation counter analysis and acceleration mass spectrometer analysis. When the carbonate is enriched with ¹³C, it can be preferred to use mass spectrometry or infra red spectroscopy. When the carbonate is enriched with ¹⁴C, it can be preferred to use scintillation counter analysis and acceleration mass spectrometer analysis.

Preferably, the insoluble carbonate is a metal carbonate. Preferably, the insoluble carbonate formulation includes at least one of calcium carbonate, magnesium carbonate and zinc carbonate. Mixtures of carbonates could be used.

An insoluble carbonate, such as calcium carbonate, is a non-absorbable chemical compound that neutralises acids such as hydrochloric acid. Hydrochloric acid occurs naturally within the stomach. For example, hydrochloric acid reacts with calcium carbonate to produce calcium chloride, water and carbon dioxide. When calcium carbonate is added to hydrochloric acid, neutralisation occurs quickly and effectively and the amount of carbon dioxide that is released is equivalent to the amount of acid that has been neutralised. The same reaction occurs with any carbonate. Thus other insoluble carbonates will also give the same results and any physiologically acceptable insoluble carbonate may be used in the method of the present invention.

Preferably, the water-insoluble carbonate contains at least about 1 atom %, more preferably at least about 5 atom %, especially at least about 10 atom %, of the selected isotope.

The natural isotopic abundance of carbon isotopes is approximately 98.93 atom % ¹²C and 1.07 atom % to ¹³C. Thus an insoluble carbonate containing an amount of ¹³C relative to ¹²C in excess of 1.07 atom % is enriched in ¹³C. The amount of the isotope that is included to in the insoluble carbonate will be selected so as to provide an adequately strong signal when the isotope content is measured. High isotope contents can give rise to disadvantages of high cost and difficulty in accurate measurement. It will often therefore be preferred for the content of ¹³C in the carbonate to be not more than about 20 atom %, more preferably not more than about 10 atom %.

The present invention also envisages the possibility of using ¹⁴C in place of ¹³C in the method of the present invention. Thus, in each of the aspects and embodiments of the invention described above in relation to ¹³C, the ¹³C can be replaced by ¹⁴C with equal utility. The only consequential change to the method described for ¹³C resides in the nature of the analytical method employed that is used to determine the ratio of excreted ¹⁴C to background ¹⁴C in the carbon dioxide. In this case, when using ¹⁴C in place of ¹³C it is be necessary to use a scintillation counter or acceleration mass spectrometer for analysis of the exhaled breath sample. It is also envisaged that isotope contents towards the lower end of the ranges referred to above will be appropriate when the carbonate is enriched with ¹⁴C.

In an embodiment, the formulation is preferably selected from the group comprising: a tablet, a capsule and a lozenge. The formulation can be in the form of a powder or a suspension.

Preferably, the formulation is a unit dosage. More preferably it is a unit dosage of at least 250 mg, especially at least about 500 mg.

Preferably, the total quantity of the insoluble carbonate that is administered to the patient is at least about 5 g, more preferably at least about 7.5 g, especially at least about 10 g, for example at least about 12 g. Some patients might require administration of a larger total quantity of insoluble carbonate, for example of at least about 15 g, or at least about 20 g. Preferably, the insoluble carbonate is administered to the patient at a rate of at least about 3 g.h⁻¹, more preferably at least about 5 g.h⁻¹. These quantities of carbonate are measured absolutely, so that the weights of other components of the formulation such as binders and capsule enclosure materials etc are not included. Such quantities of the carbonate will generally be such that the carbonate is present in the stomach in excess relative to stomach acid.

¹³C is a naturally occurring cold (not radioactive) isotope which exists in small quantities in the body. After ingestion of an enriched compound it is substantially eliminated within 12 hours and the levels return to normal.

The reaction between the insoluble carbonate and acid within the stomach causes the acid to be neutralised. Neutralisation of acid within the stomach stimulates the mucosa of the stomach to produce gastrin, a hormone that induces further secretion of hydrochloric acid.

In the presence of excess calcium carbonate this further hydrochloric acid will also be neutralised so inducing further secretion. The amount of carbon dioxide produced during the period of the test is equivalent to the amount of acid secreted over the same period.

Carbon dioxide produced within the stomach as a consequence of neutralisation as described above is absorbed into the blood stream. It then participates with exchange processes in the lungs and is excreted in the breath, together with carbon dioxide which is produced as a result of metabolic activity within the body. The present invention overcomes the problem of how to identify the carbon dioxide that has been produced as a result of the neutralisation of gastric acid following administration of the insoluble carbonate as opposed to the carbon dioxide produced by metabolism.

The time taken for the isotope content in exhaled carbon dioxide to stabilise depends on the reactions within the stomach, in particular the rate at which acid is secreted in response to the production of gastrin, and on the time taken for the carbon dioxide which is generated in the stomach to reach a steady state concentration in the blood. It will often take 60 minutes or more for the isotope content in the exhaled carbon dioxide to stabilise, and sometimes at least about 120 minutes. Accordingly, it can be preferred for the time period between the steps of (i) the initial administration of the substance or formulation containing the water-insoluble carbonate, and (ii) obtaining the sample of exhaled air after allowing the content of the or each selected isotope in the exhaled carbon dioxide to stabilise, is at least about 60 minutes, preferably at least about 120 minutes, for example at least about 150 minutes.

It can be preferred for the method of the invention to include a further step a further step (e) involving repeating steps (c) and (d) to assess whether the content of the or each selected isotope in the exhaled carbon dioxide has stabilised.

It can be preferred for the method of the invention to include at least two steps of administering a substance or formulation containing the water-insoluble carbonate to the mammal. Preferably, the time period between successive administration steps is at least about 5 minutes, more preferably at least about 10 minutes. Repeating the administration step can ensure that carbonate is present in the stomach in excess. Preferably, the quantity of the water-insoluble carbonate that is administered to the mammal is known. This can help to ensure that the administered carbonate is present in the stomach in excess.

The method of the present invention involves administering an oral dose of an insoluble carbonate (labelled with one or more isotopes). At periodic intervals the subject breathes out through a straw into a collection vessel and the expired air is measured to determine the content of the selected isotope.

The subject should preferably be fasted when performing a determination of gastric acid secretion. The reason for this is that there is a difference in human gastrointestinal physiology between the fasted and fed states. The same is true of other mammals.

The differences between the fed and fasted states are significant, and do not trivially default to just the presence or absence of food in the stomach. The myoelectric activity and motility of the stomach are very different in the fasted and fed states. In addition to fasted/fed differences, gastric emptying differs for solutions and for food and for dosage forms of various sizes. In general, gastric emptying is slower in the fed state than in the fasted state. In general, large undigestable objects (and slowly disintegrating dosage forms), for example with a particle size (diameter) of 7 mm or more, are emptied more slowly than smaller objects, for example with a particle size of 4 mm, while liquids are emptied more quickly than either of these.

For example, in the fasted state, large undigested objects (including non-disintegrated dosage forms) do not exit the stomach until the occurrence of Phase III of the migrating myoelectric complex (MMC), also known as the housekeeper phase or housekeeper wave, which empties the stomach of undigested material. In the fasted state, solutions and suspensions of small particles exit the stomach essentially continuously, and do not need to wait for a housekeeper wave, which occurs in humans approximately every 60 to 90 minutes.

The repetitive MMC cycle stops when food is ingested, and the motility pattern of the stomach changes significantly. After ingestion of a meal, the stomach grinds the meal contents slowly down to small particles, aided by the action of gastric acid and digestive enzymes. Small particles and food (or drug) in solution move through the pyloric valve into the duodenum. The presence of food materials particularly fatty acids and amino acids) in the duodenum after the initiation of eating results in triggering this “fed gastric state”, which is characterized by slower gastric emptying and the absence of MMCs. Larger objects (undigested food pieces, non-disintegrated dosage forms) do not exit the stomach until the entire meal has been broken down to small particles which can pass the pylorus, and the GI system senses that material left in the stomach is not digestible.

In addition to the effects discussed above, the pH of the stomach increases to around pH 5 when a meal is taken, and then falls back to about pH 2 in about 2 hr. Furthermore, food may buffer acid that has been secreted by the stomach, a proportion of the acid therefore not being available for neutralisation by the carbonate.

In the context of the present invention, fasted means that the patient has not consumed food within a period of at least 4 hours. Preferably no food has been consumed for at least 8 hours before commencement of the method, and more preferably the period of fasting has been at least overnight (i.e. at least about 12 hours) before the test is performed. However, consumption of a small amount of food, or water, whilst not ideal can be tolerated provided that it has an insignificant effect on gastric motility or pH.

The method of the invention can be performed in a fed patient to determine the change in acid secretion due to ingestion of a measured amount of food provided that the test meal has been assessed in vitro for its buffering activity.

The time period i.e. interval between the administration of successive dosages of the carbonate may be the same or it may vary. Similarly the time period (i.e. interval) between the collection of successive samples of breath may be the same or it may vary.

Preferably the administration step or the determining step or each of them is repeated at least 2 times, more preferably at least 3 times, and further preferably at least 5 times. Ten or more repeats could be made if desired.

The time interval between successive dosages and the time interval between the collection of successive breath samples may be the same as each other or these may be different.

Preferably, the interval between successive dosages is from 1 to 30 minutes, and more preferably is at least about 5 minutes, more preferably at least about 10 minutes.

Preferably, the time period between the collection of successive breath samples is from 1 to 600 minutes, and more preferably is about 15 minutes.

Preferably, the method includes the step of monitoring the variation in the content of the selected or each selected isotope in the exhaled carbon dioxide with time, for example by plotting the variation in a graph.

The results from the determining against time plot are correlated with actual amounts of gastric acid secretion as follows. The scales used in the plots represent the change in the isotope content relative to a baseline level. When the selected isotope is ¹⁴C, the baseline level can be measured relative to background ¹⁴C level. When the selected isotope is ¹³C, the baseline level can be determined by measuring the ¹³C content before administration of enriched carbonate. The change in the isotope content with time will tend after time to reflect the amount of carbon dioxide produced as a result of neutralisation of gastric acid, once a steady state has been reached with regard to stomach reactions and absorption into the patient's blood.

In order to identify the actual amount of gastric acid secretion it is first necessary to convert these figures to give the actual amount of carbon dioxide produced. This can involve collection of breath in a bag and then analysis of the CO₂ content. CO₂ analysis could also be performed using a flow system or other techniques.

It is possible that not all of the carbon dioxide produced in the body is excreted in the breath and a small percentage may be lost elsewhere, such as in the urine and by diffusion into a slowly equilibrating volume. This will depend on the individual patient and could be the subject of further research but recovery of carbon dioxide may be assumed to be 80% for the purposes of this test.

When the selected isotope is ¹³C, an expression can be derived relating the following quantities: ¹³C/¹²C ratio in breath before administration of carbonate (baseline ratio), ¹³C/¹²C ratio in breath after administration of carbonate, and ¹³C/¹²C ratio in the carbonate. The increase in ¹³C in the breath after administration of carbonate is due to the presence of ¹³C from enriched carbonate neutralised by gastric acid.

The total amount of carbon dioxide in the breath can be measured by collection into a Douglas Bag. This will consist of the carbon dioxide due to normal metabolism, plus the carbon dioxide generated from the neutralisation of ingested carbonate by gastric acid. Using the expression relating the various ratios, we can then calculate the amount of carbon dioxide in the bag, in moles, which is derived from the neutralised carbonate.

In order to relate the result of this calculation to actual gastric acid secretion, it is necessary to make some assumptions.

Firstly, is should be assumed that the ¹³C/¹²C ratio in breath before administration of carbonate (baseline ratio) does not vary with time. This variation can safely be treated as negligible in the context of this test. Secondly, although the amount of carbon dioxide in the breath due to normal metabolism can vary significantly, this variation will be corrected for if the Douglas Bag collection is made immediately before or after the collection for ¹³C/¹²C ratio from which the calculation of acid secretion is to be made. Thirdly, it has to be assumed that a proportion of the carbon dioxide generated by acid neutralisation is also not excreted in the breath.

Subsequent calculations are based on the reaction in the stomach involving:

2HCl+CaCO₃→CO₂+H₂O+CaCl₂,

so that the ratio of the number of moles of exhaled carbon dioxide to the number of moles of reacting acid is equal to 2.0, subject to a correction for carbon dioxide that is not excreted in the breath.

### EXAMPLE 1

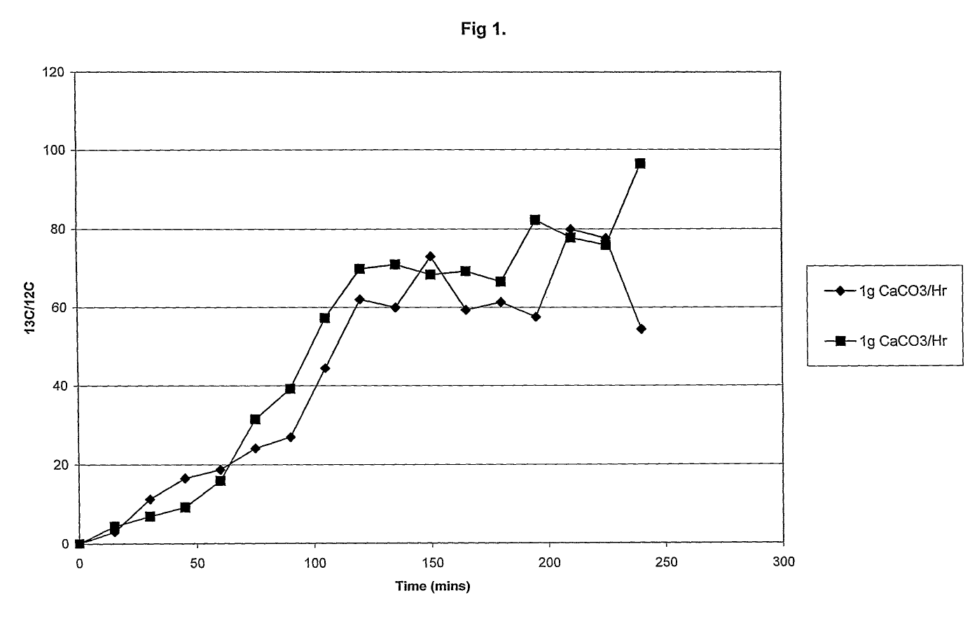

A human volunteer was given an oral dose of calcium carbonate labelled with 10 atom % of ¹³C every 15 minutes over a period of 4 hours. The oral dose contained 250 mg of calcium carbonate. At 10 minute intervals, samples of exhaled (expired) air were obtained from the subject by breathing into a suitable collection vessel such as a test tube. The expired air was subjected to mass spectrometry to measure the ratio of ¹³C to ¹²C in the exhaled carbon dioxide. The results are illustrated in FIG. 1.

The ratios plotted in FIG. 1 show the results of two experiments using ¹³C labelled calcium carbonate and demonstrate a rise in ¹³C that begins to plateau at about 120 minutes. The time at which the plateau is reached might vary from patient to patient.

Note that there is a rise in the excretion of carbon dioxide over approximately the first 120 minutes, with a plateau occurring after that. The observed plateau could arise because of the reactions in the stomach, the need for the carbon dioxide in the stomach to equilibrate with blood and for the carbon dioxide in the blood to equilibrate with carbon dioxide in the lungs. Previous work has suggested that there might also be an exchangeable pool of carbon dioxide within the body.

Accordingly, it is believed that the plateau occurs when ¹³CO₂ excreted through the breath is approximately equivalent to the amount of acid that is neutralised in the stomach less that amount which is excreted by the urine or sequestered in a non or slowly exchangeable compartment. It is important in order to obtain accurate results that the determination of the content of the selected isotope in exhaled carbon dioxide is performed when it has stabilised, for example by making repeated determinations from successive samples of exhaled air.

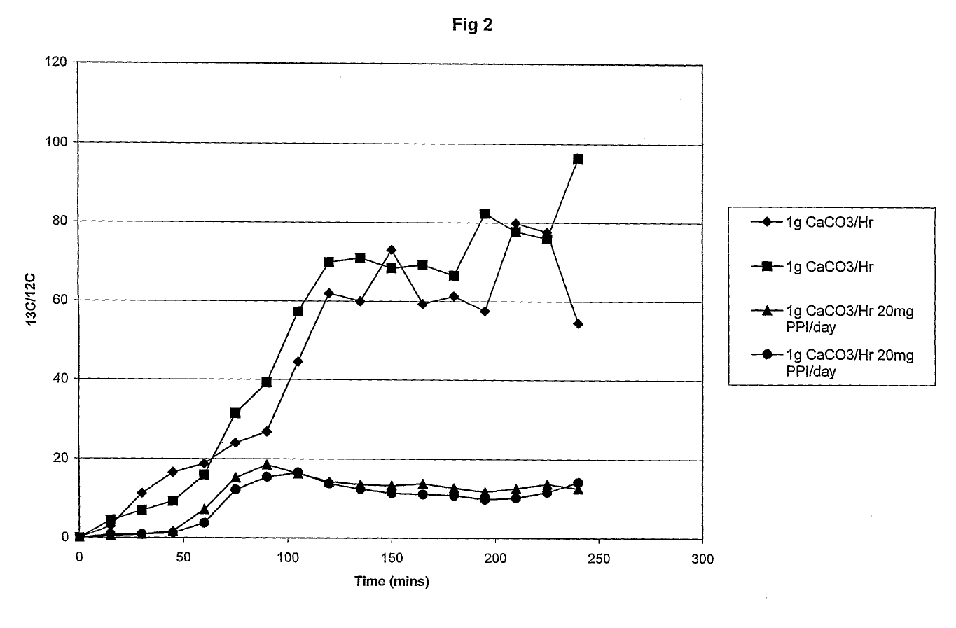

In a second experiment the subject was treated with omeprazole 20 mg daily for two weeks and the experiment repeated twice under otherwise identical conditions. FIG. 2 shows that the amount of ¹³C labelled carbon dioxide was substantially reduced in this case as compared with the original experiments.

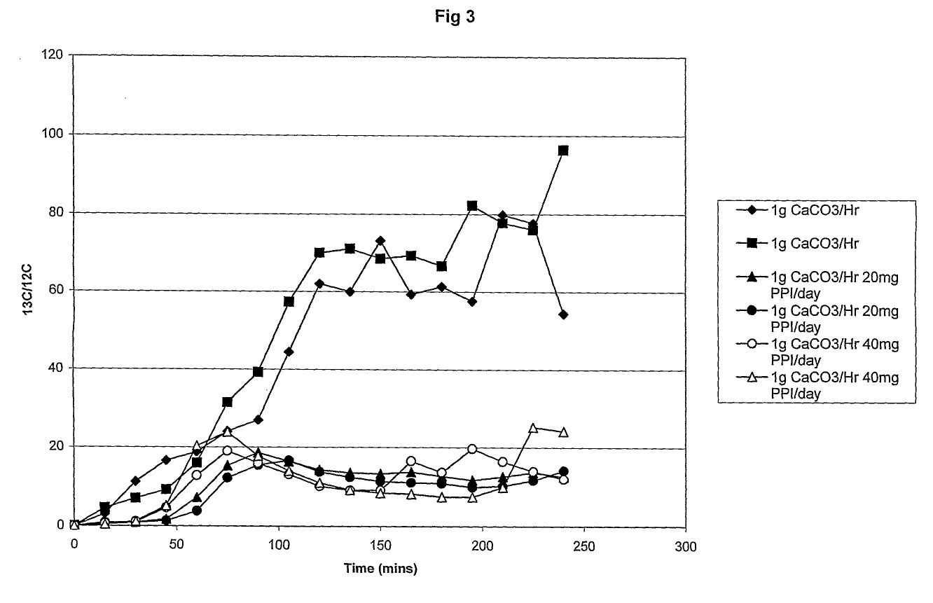

The subject then did two further experiments again under otherwise identical conditions but now including a daily dosage of 40 mg of omeprazole. FIG. 3 shows similar results.

This finding is consistent with the observation that 20 mg of omeprazole orally in most individuals produces maximal acid suppression for this class of drug so the use of 40 mg does not confer an advantage in this subject.

### EXAMPLE 2

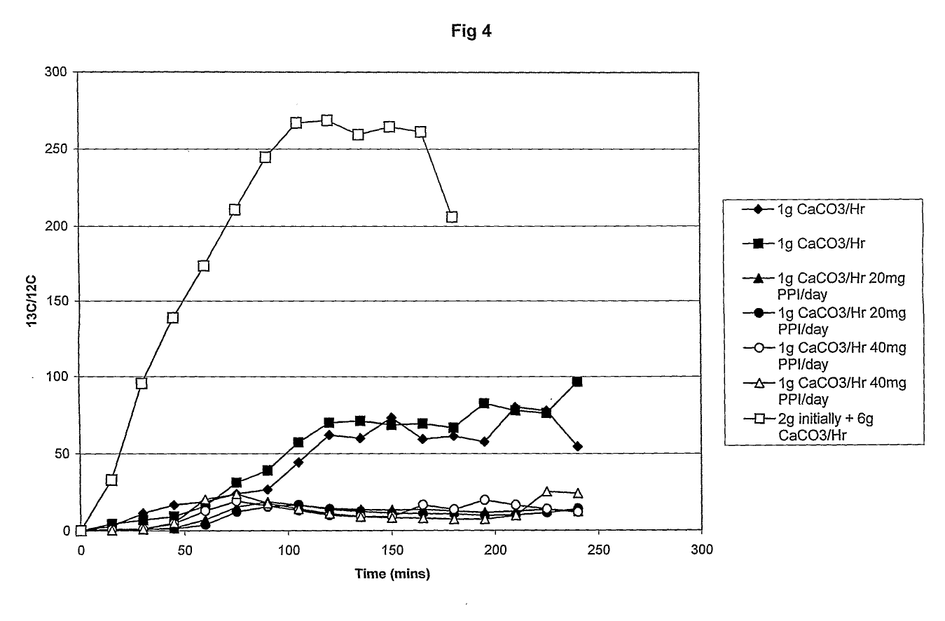

A human volunteer was given an initial oral dose of 2 g of calcium carbonate labelled with 10 atom % of ¹³C then a further 500 mg dose of the calcium carbonate every 5 minutes over a period of 3 hours. At 15 minute intervals, samples of exhaled (expired) air were obtained from the subject by breathing into a suitable collection vessel such as a test tube. The expired air was subjected to mass spectrometry to measure the ratio of ¹³C to ¹²C in the exhaled carbon dioxide. The results are illustrated in FIG. 4.

The ratios plotted in FIG. 4 show the results of two experiments using ¹³C labelled calcium carbonate and demonstrate a rise in ¹³C that begins to plateau at about 120 minutes. The time at which the plateau is reached might vary from patient to patient.

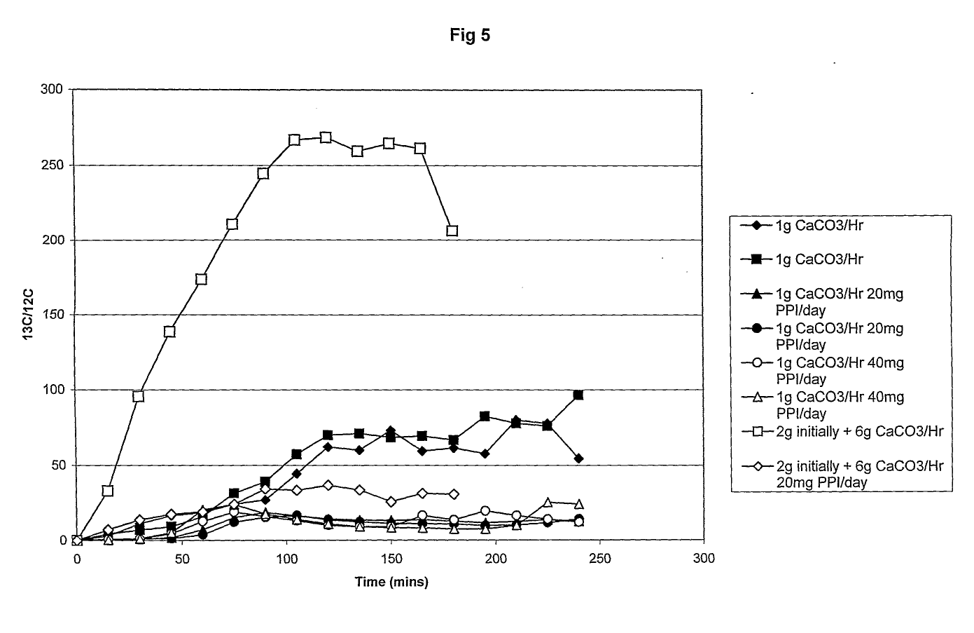

In a second experiment the subject was treated with 20 mg omeprazole daily for one week and the experiment repeated under otherwise identical conditions. FIG. 5 shows that the amount of ¹³C labelled carbon dioxide was substantially reduced in this case as compared with the original experiments.

Further refinements to the method of the invention as exemplified above are possible.

The above data is based on only one subject and further refinements would involve a wider study. The Figures do not take into account variations that may have occurred in the production of carbon dioxide within the general metabolism of the subject. It may be that there will be differences in excretion and sequestration from one patient to another.

Alkaline stimulated acid secretion has not, in the past, been studied, test meals or the injection of secretagogues have been used instead. Further experimental work using more than one patient will allow an assessment in molar terms of the relationship between carbon dioxide excretion in the breath and the amount of acid secreted.

By comparing a sufficient number of differing individuals it will be possible to establish a normal range for alkaline stimulated acid secretion and to confirm the effects of pharmacological acid suppression.

One potentially useful application, which has not previously been possible, will involve performing experiments on subjects with pernicious anaemia in whom there is no acid secretion in the stomach whatsoever. This will demonstrate whether or not there is any “leakage” of ¹³C across the gastrointestinal mucosa and into the breath.

The above Examples and the related FIGS. 1 to 5 show significant and substantial differences in the test result that mirrors gastric acid output in one subject. This confirms that the technique can detect a difference in acid secretion when acid suppressive therapy is being used.

Table 1 contains the data sets from which each of the Figures were generated. The dosages of calcium carbonate and acid suppressing drug (if taken) for each experiment are described in the text and in the legends for each graph

It is also possible to measure total carbon dioxide excretion in addition to ¹³C/¹²C ratios. This is expected to provide a more consistent result. Similarly it is possible to measure plasma gastrin to determine what degree of stimulus is provided by the ingestion of calcium carbonate.

The test could be used for general use in epidemiological research, in research into the physiology of gastric acid secretion and for research within the pharmaceutical industry for the identification of newer and more effective acid suppressants. This test will be particularly valuable in clinical management, especially in those individuals who fail to respond to acid suppression. It may be used to identify individuals who fail to respond to acid suppressants and in whom a supra-normal dose is required. It can also be used to detect those in whom acid suppression has already been achieved and further medication is unlikely to be of benefit. There may be a small number of individuals in whom the medication does not have any effect at all and this method allows identification of those individuals.

1. A method of measuring the amount of gastric secretion in a mammal, the method comprising the steps:
a. administering a substance or formulation containing an excess quantity of a water-insoluble carbonate to the mammal to react with acid in the stomach, in which the insoluble carbonate is enriched with at least one isotope selected from ¹³C, ¹⁴C, ¹⁷O and ¹⁸O in a known amount,
b. allowing the content of the or each selected isotope in the exhaled carbon dioxide to stabilise,
c. obtaining a sample of exhaled air containing carbon dioxide, and
d. determining the content of the selected or each selected isotope in the exhaled carbon dioxide.

2. A method as claimed in claim 1, in which the step of determining the isotope content involves determining the ratio of ¹³C to ¹²C.

3. A method as claimed in claim 1, in which the step of determining the isotope content involves determining the ratio of ¹⁴C in exhaled carbon dioxide to background ¹⁴C.

4. A method as claimed in claim 1, which includes a step, prior to the step of administering the carbonate, of obtaining a sample of exhaled air containing carbon dioxide and determining the content of the selected or each selected isotope in the exhaled carbon dioxide.

5. A method as claimed in claim 1, which includes a further step (e) involving repeating steps (c) and (d) to assess whether the content of the or each selected isotope in the exhaled carbon dioxide has stabilised.

6. A method as claimed in claim 1, which includes at least two steps of administering a substance or formulation containing the water-insoluble carbonate to the mammal.

7. A method as claimed in claim 6, in which the time period between successive administration steps is at least about 5 minutes.

8. A method as claimed in claim 1, in which the time period between the steps of (i) the initial administration of the substance or formulation containing the water-insoluble carbonate, and (ii) obtaining the sample of exhaled air after allowing the content of the or each selected isotope in the exhaled carbon dioxide to stabilise, is at least about 60 minutes.

9. A method as claimed in claim 1, in which the quantity of the water-insoluble carbonate that is administered to the mammal is known.

10. A method as claimed in claim 1, in which the insoluble carbonate comprises at least one of calcium carbonate, magnesium carbonate and zinc carbonate.

11. A method as claimed in claim 1, in which the mammal is a human.

12. A method as claimed in claim 1, which includes the step of plotting the content of the selected or each selected isotope in the exhaled carbon dioxide against time.

13. A method as claimed in claim 1, in which the water-insoluble carbonate contains at least about 1 atom % of the selected isotope.

14. A method as claimed in claim 1, in which the substance or formulation does not include any component other than the water-insoluble carbonate which reacts with acid in the stomach.

15. A method as claimed in claim 1, in which the total quantity of the insoluble carbonate that is administered to the patient is at least about 5 g.

16. A pharmaceutically acceptable formulation containing a water-insoluble carbonate which can react with acid in a mammalian stomach, in which the insoluble carbonate is enriched with at least one isotope selected from ¹³C, ¹⁴C, ¹⁷O and ¹⁸O in a known amount, in which the formulation does not include any component other than the water-insoluble carbonate which reacts with acid in the stomach.

17. A formulation as claimed in claim 16, in which the formulation is provided in the form of one of a tablet, a drink, a capsule and a lozenge.

18. A formulation as claimed in claim 16, wherein the formulation is provided in the form of a powder or a suspension.

19. A formulation as claimed in claim 16, wherein the formulation is a unit dosage.

20. A formulation as claimed in claim 19, wherein the unit dosage is at least 250 mg.

21. A formulation as claimed in claim 16, in which the water-insoluble carbonate contains at least about 1 atom % of the selected isotope.

22. (canceled)

23. (canceled)
